(19) United States
(12) Patent Application Publication
(10) Pub. No.: US 2015/0056605 A1
(43) Pub. Date: Feb. 26, 2015

(54) IDENTIFICATION OF BLOOD BASED METABOLITE BIOMARKERS OF PANCREATIC CANCER

(71) Applicant: PURDUE RESEARCH FOUNDATION, West Lafayette, IN (US)

(72) Inventors: M. Daniel Raftery, Seattle, WA (US); Vincent M. Asiago, Johnston, IA (US); Kwadwo Owusu-sarfo, Morristown, NJ (US); Bowei Xi, West Lafayette, IN (US)

(73) Assignee: PURDUE RESEARCH FOUNDATION, West Lafayette, IN (US)

(21) Appl. No.: 14/465,535

(22) Filed: Aug. 21, 2014

Related U.S. Application Data
(60) Provisional application No. 61/868,398, filed on Aug. 21, 2013.

(51) Int. Cl. G01N 33/574; G01N 33/68; G01N 24/08; H01J 49/40; H01J 49/00; C12Q 1/00; G01N 30/72

(52) U.S. Cl. CPC G01N 33/57438; C12Q 1/00; G01N 33/6848; G01N 30/7233; H01J 49/40; H01J 49/004; H01J 49/0036; G01N 24/08; G01N 2560/00
USPC 435/4; 436/64; 250/282; 324/309

(57) ABSTRACT

The present disclosure relates to a panel of a plurality of metabolite species that is useful for the identification or detection of subjects having pancreatic cancer, including methods for identifying such metabolic biomarkers within biological samples. The disclosure also includes a statistical model for predicting the presence of pancreatic cancer in a subject's biofluid by quantifying and comparing positive and negative fold changes in metabolite species' concentration; comparing the subject's metabolite species' concentrations to a predetermined value.

### RELATED APPLICATIONS

The present application claims the benefit of U.S. provisional application Ser. No. 61/868,398, filed Aug. 21, 2013, the contents of which are incorporated by reference herein in its entirety.

### TECHNICAL FIELD

The present disclosure generally relates to small molecule metabolic biomarkers. In particular, the present disclosure relates to a panel of metabolite species that is useful for the identification of subjects having pancreatic cancer, including methods for identifying such metabolic biomarkers within biological samples.

### BACKGROUND

Pancreatic cancer (PC) afflicts both men and women, and is the fourth leading cause of cancer related deaths in the United States. The overall 5-year survival rate of patients with PC is dismal, with 95% of patients dying within five years of diagnosis. According to National Cancer Institute statistics, approximately 44,000 men and women will be diagnosed with PC and 37,000 are expected to die of this disease in 2012. The number of PC deaths is projected to increase by 55% by the year 2030 due in part to PC's poor prognosis.

PC is asymptomatic until late in the disease process and thus, late diagnosis leads to the alarmingly high mortality rate. Specific causes for the development of PC are unknown. Major risk factors include age, smoking, diabetes, pancreatitis, obesity, and lack of physical activity.

Tests using computed tomography (CT) scans, ultrasonography, endoscopic retrograde cholangiopancreatography (ERCP), percutaneous transhepatic cholangiography (PTC) and biopsy are often used to assist in the diagnosis of PC. However, the inaccessibility of the pancreas due to its deep anatomical location makes examination by available physical or radiological means ineffective. Resection, or the removal of the affected area, remains the best possibility for survival but this approach is unfortunately restricted to late stage PC because of the challenges involved in early detection.

Previous studies have been focused on the identification of molecular markers as a more reliable approach for detecting PC. Many blood markers, including cancer antigen (CA19-9), carcinoembryonic antigen-related cell adhesion molecule 1 (CEACAM1), macrophage inhibitory cytokine 1 (MIC1), carcinoembryonic antigen (CEA), alphafetoprotein (AFP), DU-PAN-2, alpha4GnT, cytokeratin-19 (CK-19) mRNA, and tissue polypeptide antigen have been examined for utility in the early detection of pancreatic cancer. Unfortunately, the use of these markers does not provide the required sensitivity and specificity for routine screening. There is no reliable screening tool for early detection of pancreatic cancer either in the general population or in at-risk patient populations that is currently available.

An alternative approach is metabolomics, a fast growing area in systems biology that combines data-rich analytical techniques such as nuclear magnetic resonance (NMR) spectroscopy and/or mass spectrometry (MS), with chemometrics, and promises the identification of sensitive metabolite biomarkers associated with disease, drug treatment, toxicity and environmental effects among its many applications. Metabolites are the downstream products of genes, transcripts and protein functions in biological systems and they can be especially sensitive to perturbations in a number of metabolic pathways and varied pathological conditions. Recent advances in cancer biomarker discovery promise development of early disease diagnostics as well as understanding perturbed metabolic pathways. To date, a few metabolomics investigations have focused on the identification of metabolite biomarkers for PC using samples from animal models or humans. These studies have analyzed urine, tissue, blood serum/plasma or saliva metabolic profiles using NMR or MS methods. Notably, each study, owing to the combination of the metabolic complexity of different biological samples and the varied sensitivity, selectivity, or resolution associated with each type of analytical method, has identified different set of distinguishing metabolites.

### SUMMARY OF THE INVENTION

The present disclosure relates to a panel of metabolite species that is useful for the identification of subjects having pancreatic cancer, including methods for identifying such metabolic biomarkers within biological samples.

In one aspect, the disclosure includes a method comprising measuring the concentration of at least two metabolite species in a sample of a biofluid from a subject having pancreatic cancer, wherein the metabolite species is a component of a panel of a plurality of metabolite species, wherein a change in the concentration of the metabolite species is useful for the identification of subjects having pancreatic cancer. In certain embodiments the concentration of the metabolite species is normalized. In preferred embodiments, the method includes the step of comparing the measured concentration of the metabolite species to a predetermined value calculated using a model based on concentrations of a plurality of the metabolic species that are components of the panel.

In certain embodiments, the panel of metabolite species comprises two to nine compounds selected from the group consisting of alanine, creatinine, formate, glucose, glutamate, glutamine, histidine, lactate, valine, and mixtures thereof. In preferred embodiments, the panel consists of alanine, creatinine, formate, glucose, glutamate, glutamine, histidine, lactate, and valine.

In general, the panel comprises metabolite species that have been identified by at least one of the methods selected from nuclear magnetic resonance (NMR) spectroscopy, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry. In certain embodiments, the panel comprises metabolite species that have been identified by nuclear magnetic resonance (NMR) spectroscopy. In some embodiments, the panel comprises metabolite species that have been identified by liquid chromatography-mass spectrometry (LC-MS). Typically, the biofluid is selected from the group consisting of blood, plasma, serum, sweat, saliva, sputum, and urine. In preferred embodiments, the biofluid is serum.

In other aspects, a panel of metabolite species is disclosed that comprises a plurality of metabolite species selected from the group consisting of alanine, creatinine, formate, glucose, glutamate, glutamine, histidine, lactate, valine, and mixtures thereof. In certain embodiments, the panel consists of alanine, creatinine, formate, glucose, glutamate, glutamine, histidine, lactate, and valine. In some embodiments, a diagnostic cassette comprises reagents for the detection of the metabolite species of such a panel.

Also disclosed is a kit for the analysis of a sample of a biofluid of a subject, comprising aliquots of standards of each compound of a panel of metabolite species; an aliquot of an internal standard; and an aliquot of a control biofluid. Typically the control biofluid is serum from a control source that is conspecific with the subject. In some embodiments, the panel consists of alanine, creatinine, formate, glucose, glutamate, glutamine, histidine, lactate, and valine. Typically, the kit includes instructions for use.

### BRIEF DESCRIPTION OF THE DRAWINGS

The above-mentioned aspects of the present teachings and the manner of obtaining them will become more apparent and the teachings will be better understood by reference to the following description of the embodiments taken in conjunction with the accompanying drawings, in which corresponding reference characters indicate corresponding parts throughout the several views.

FIG. 1 is a schematic representation of the data analysis protocols for the development of cross-validated partial least squares discriminant analysis (PLS-DA) prediction model and its validation.

FIG. 2A shows a ¹H NMR spectrum, obtained by averaging the spectra of pancreatic cancer samples in the training set. FIG. 2B shows a difference spectrum between the average spectrum of the pancreatic cancer and healthy controls from the training sample set. The numbered arrows indicate glutamate (1); formate (2), glucose (3), lactate (4), creatinine (5), alanine (6), glutamine (7), histidine (8), and valine (9).

FIG. 3A-FIG. 3I show box and whisker plots that show a comparison of concentration of selected metabolic biomarkers including formate, FIG. 3A, histidine, FIG. 3B, glucose, FIG. 3C, lactate, FIG. 3D, creatinine, FIG. 3E, glutamine, FIG. 3F, glutamate, FIG. 3G, alanine, FIG. 3H, and valine, FIG. 3I. Box-and-whisker plots showing the distribution of relative concentrations of the metabolites are used for model building, in pancreatic cancer and normal subjects from the training set. The middle horizontal line in the box represents the median, the bottom and top boundaries represent the 25^{th} and 75^{th} percentiles, respectively. The lower and upper whiskers represent the 5^{th} and 95^{th} percentiles, respectively, and the open circles represent outliers.

FIG. 4A shows a PLS-DA score plot for the statistical model developed and cross-validated using a training set of 87 (55 pancreatic cancer and 32 healthy control) samples, FIG. 4B shows a receptor operating curve (ROC) for the PLS-DA prediction model. FIG. 4C shows Box-and-whisker plot of the predication scores for the two sample classes.

FIG. 5A shows the score plot for the validation set of samples obtained from the PLS-DA predication model; FIG. 5B shows the ROC curve generated from the PLS-DA prediction model; FIG. 5C shows a Box-and-whisker plot for the two sample classes showing discrimination between normal and pancreatic cancer patient samples using the predicted scores.

FIG. 6 shows the receiver operating characteristic (ROC) space showing Monte Carlo Cross Validation (MCCV) (300 iterations) results of PLS-DA models on 9 biomarkers to discriminate pancreatic cancer samples from healthy controls. Each diamond represents an iteration of the true model; each square represents a permutation model.

FIG. 7 shows a summary of the altered metabolic pathways associated with the metabolites that showed significant statistical differences between pancreatic cancer and control samples. The metabolites indicated with solid borders, formate, glucose, glutamate, and lactate showed an increase in concentration in pancreatic cancer patients while those with dashed borders, alanine, creatinine, glutamine, histidine, and valine showed a decrease in concentration.

### DETAILED DESCRIPTION OF EXEMPLARY EMBODIMENTS

Serum metabolite profiles in pancreatic cancer (PC) patients (n=78) and non-disease controls (n=48) were measured using ¹H nuclear magnetic resonance (NMR) spectroscopy with a focus on the identification of metabolite biomarkers associated with PC pathology and testing the classification accuracy of the statistical model developed using the metabolite data. Nine distinguishing metabolites (alanine, citrate, creatinine, formate, glucose, glutamine, histidine, lactate, and valine) were identified from the univariate and multivariate logistic regression analysis of the NMR data from one batch of samples (55 from PC subjects; 32 from healthy control subjects). A cross-validated regression model built using these metabolites differentiated the cancer and control groups with a high accuracy and an area under the receiver operating characteristic curve (AUROC) of 0.94. This model was validated using the NMR data from an entirely different set of samples (23 from PC subjects; 16 from healthy control subjects) which showed similar performance of the model with an AUROC of 0.86.

In this study, serum metabolite profiling was performed to identify potential metabolic biomarker candidates that can identify subjects having pancreatic cancer. The demonstrated ability to distinguish pancreatic cancer patients from healthy controls demonstrates the utility of the serum metabolites based regression model to identify patients with pancreatic cancer.

Unless defined otherwise, all technical and scientific terms used herein have the meaning commonly understood by a person skilled in the art to which this invention belongs. Numbers in scientific notation are expressed as product of a coefficient between 1 and 10 and an exponential multiplier, ten raised to an integer power (e.g., 9.6×10⁻⁴), or abbreviated as the coefficient followed by “E,” followed by the exponent (e.g., 9.6E-04).

As used herein, “metabolite” or “metabolite biomarker” refers to any substance produced or used during all the physical and chemical processes within the body that create and use energy, such as: digesting food and nutrients, eliminating waste through urine and feces, breathing, circulating blood, and regulating temperature. The term “metabolic precursors” refers to compounds from which the metabolites are made. The term “metabolic products” refers to any substance that is part of a metabolic pathway (e.g. metabolite, metabolic precursor). The term “metabolite species” as used herein refers to an identified molecule or an identified molecular moiety, such as a lipid alkyl moiety, that is detectable by the measurement technique that is used. For further information, please see U.S. patent application publication US 2007/0221835 the contents of which are incorporated herein by reference in its entirety.

As used herein, “biological sample” refers to a sample obtained from a subject. In preferred embodiments, biological sample can be selected, without limitation, from the group of biological fluids (“biofluids”) consisting of blood, plasma, serum, sweat, saliva, including sputum, urine, and the like. As used herein, “serum” refers to the fluid portion of the blood obtained after removal of the fibrin clot and blood cells, distinguished from the plasma in circulating blood. As used herein, “plasma” refers to the fluid, non-cellular portion of the blood, as distinguished from the serum, which is obtained after coagulation.

As used herein, “subject” refers to any warm-blooded animal, particularly including a member of the class Mammalia such as, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female. “Conspecific” means of or belonging to the same species, and when used as a noun, a member of the same species.

As used herein, “normal control subjects” or “normal controls” means healthy subjects who are clinically free of cancer. “Normal control sample” or “control sample” refers to a sample of biofluid that has been obtained from a normal control subject. A normal control sample or a control sample is preferably obtained from a conspecific of the test subject. The normal control subjects were used to help determine a predetermined value.

As used herein, “pancreatic cancer” is intended to encompass all forms of mammalian pancreatic carcinomas, sarcomas, and melanomas which occur in the poorly differentiated, moderately differentiated, and well differentiated forms.

As used herein, “detecting” refers to methods which include identifying the presence or absence of substance(s) in the sample, quantifying the amount of substance(s) in the sample, and/or qualifying the type of substance.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

It is to be understood that this invention is not limited to the particular component parts of a device described or process steps of the methods described, as such devices and methods may vary. It is also to be understood that the terminology used herein is for purposes of describing particular embodiments only, and is not intended to be limiting. As used in the specification and the appended claims, the singular forms “a,” “an,” and “the” include plural referents unless the context clearly indicates otherwise. The terms “comprises,” “comprising,” and the like are intended to have the broad meaning ascribed to them in U.S. Patent Law and can mean “includes,” “including” and the like.

Metabolite profiling uses high-throughput analytical methods such as nuclear magnetic resonance spectroscopy and mass spectroscopy for the quantitative analysis of hundreds of small molecules (less than ˜1000 Daltons) present in biological samples. Owing to the complexity of the metabolic profile, multivariate statistical methods are extensively used for data analysis. The high sensitivity of metabolite profiles to even subtle stimuli can provide the means to detect the early onset of various biological perturbations in real time.

One of ordinary skill in the art will recognize that these identified biomarkers can be detected by alternative methods of suitable sensitivity, such as HPLC, immunoassays, enzymatic assays or clinical chemistry methods.

In one embodiment of the invention, samples may be collected from individuals over a longitudinal period of time. Obtaining numerous samples from an individual over a period of time can be used to verify results from earlier detections and/or to identify an alteration in marker pattern as a result of, for example, pathology. In one embodiment of the invention, the samples are analyzed without additional preparation and/or separation procedures. In another embodiment of the invention, sample preparation and/or separation can involve, without limitation, any of the following procedures, depending on the type of sample collected and/or types of metabolic products searched: removal of high abundance polypeptides or proteins (e.g., albumin, and transferrin); addition of preservatives and calibrants, desalting of samples; concentration of sample substances; protein precipitation, protein digestions; and fraction collection. In yet another embodiment of the invention, sample preparation techniques concentrate information-rich metabolic products and deplete polypeptides and proteins or other substances that would carry little or no information such as those that are highly abundant in serum.

In another embodiment of the invention, sample preparation takes place in a manifold or preparation/separation device. Such a preparation/separation device may, for example, be a microfluidics device, such as a diagnostic cassette. In yet another embodiment of the invention, the preparation/separation device interfaces directly or indirectly with a detection device. Such a preparation/separation device may, for example, be a fluidics device.

In another embodiment of the invention, the removal of undesired polypeptides (e.g., high abundance, uninformative, or undetectable polypeptides) can be achieved using high affinity reagents, high molecular weight filters, column purification, ultracentrifugation and/or electrodialysis. High affinity reagents include antibodies that selectively bind to high abundance polypeptides or reagents that have a specific pH, ionic value, or detergent strength. High molecular weight filters include membranes that separate molecules on the basis of size and molecular weight. Such filters may further employ reverse osmosis, nanofiltration, ultrafiltration and microfiltration.

Ultracentrifugation constitutes another method for removing undesired polypeptides. Ultracentrifugation is the centrifugation of a sample at speeds above 20,000 rpm, and typically about 60,000 to 100,000 rpm while monitoring with an optical system the sedimentation (or lack thereof) of particles. Finally, electrodialysis is an electromembrane process in which ions are transported through ion permeable membranes from one solution to another under the influence of a potential gradient. Since the membranes used in electrodialysis have the ability to selectively transportions having positive or negative charge and reject ions of the opposite charge, electrodialysis is useful for concentration, removal, or separation of electrolytes.

In another embodiment of the invention, the manifold or microfluidics device or diagnostic cassette performs electrodialysis to remove high molecular weight polypeptides or undesired polypeptides. Electrodialysis can be used first to allow only molecules under approximately 35-30 kD to pass through into a second chamber. A second membrane with a very small molecular weight cutoff (roughly 500 Da) allows smaller molecules to exit the second chamber.

Upon preparation of the samples, metabolic products of interest may be separated in another embodiment of the invention. Separation can take place in the same location as the preparation or in another location. In one embodiment of the invention, separation occurs in the same microfluidics device where preparation occurs, but in a different location on the device. Samples can be removed from an initial manifold location to a microfluidics device or diagnostic cassette using various means, including an electric field. In another embodiment of the invention, the samples are concentrated during their migration to the microfluidics device or diagnostic cassette using reverse phase beads and an organic solvent elution such as 50% methanol. This elutes the molecules into a channel or a well on a separation device of a microfluidics device or diagnostic cassette.

Chromatography constitutes another method for separating subsets of substances. Chromatography is based on the differential absorption and elution of different substances. Liquid chromatography (LC), for example, involves the use of fluid carrier over a non-mobile phase. Conventional LC columns have an inner diameter of roughly 4.6 mm and a flow rate of roughly 1 ml/min. Micro-LC has an inner diameter of about 1.0 mm and a flow rate of about 40 μL/min. Capillary LC utilizes a capillary with an inner diameter of roughly 300 μm and a flow rate of approximately 5 μL/min. Nano-LC is available with an inner diameter of 50 μm-1 mm and flow rates of 200 mL/min. The sensitivity of nano-LC as compared to HPLC is approximately 3700 fold. Other types of chromatography suitable for additional embodiments of the invention include, without limitation, thin-layer chromatography (TLC), reverse-phase chromatography, high-performance liquid chromatography (HPLC), and gas chromatography (GC).

In another embodiment of the invention, the samples are separated using capillary electrophoresis separation. This will separate the molecules based on their electrophoretic mobility at a given pH (or hydrophobicity). In another embodiment of the invention, sample preparation and separation are combined using microfluidics technology. A microfluidic device is a device that can transport liquids including various reagents such as analytes and elutions between different locations using microchannel structures.

Suitable detection methods are those that have a sensitivity for the detection of an analyte in a biofluid sample of at least 50 μM. In certain embodiments, the sensitivity of the detection method is at least 1 μM. In other embodiments, the sensitivity of the detection method is at least 1 nM.

In one embodiment of the invention, the sample may be delivered directly to the detection device without preparation and/or separation beforehand. In another embodiment of the invention, once prepared and/or separated, the metabolic products are delivered to a detection device, which detects them in a sample. In another embodiment of the invention, metabolic products in elutions or solutions are delivered to a detection device by electrospray ionization (ESI). In yet another embodiment of the invention, nanospray ionization (NSI) is used. Nanospray ionization is a miniaturized version of ESI and provides low detection limits using extremely limited volumes of sample fluid.

In another embodiment of the invention, separated metabolic products are directed down a channel that leads to an electrospray ionization emitter, which is built into a microfluidic device (an integrated ESI microfluidic device). Such integrated ESI microfluidic device may provide the detection device with samples at flow rates and complexity levels that are optimal for detection. Furthermore, a microfluidic device may be aligned with a detection device for optimal sample capture.

Suitable detection devices can be any device or experimental methodology that is able to detect metabolic product presence and/or level, including, without limitation, IR (infrared spectroscopy), NMR (nuclear magnetic resonance spectroscopy), including variations such as correlation spectroscopy (COSy), nuclear Overhauser effect spectroscopy (NOESY), and rotating frame nuclear Overhauser effect spectroscopy (ROESY), and Fourier Transform, 2-D PAGE technology, Western blot technology, tryptic mapping, in vitro biological assay, immunological analysis, LC-MS (liquid chromatography-mass spectrometry), LC-TOF-MS, LC-QTOF, LC-MS/MS, and MS (mass spectrometry).

For analysis relying on the application of NMR spectroscopy, the spectroscopy may be practiced as one-, two-, or multidimensional NMR spectroscopy or by other NMR spectroscopic examining techniques, among others also coupled with chromatographic methods (for example, as LC-NMR). In addition to the determination of the metabolic product in question, ¹H-NMR spectroscopy offers the possibility of determining further metabolic products in the same investigative run. Combining the evaluation of a plurality of metabolic products in one investigative run can be employed for so-called “pattern recognition”. Typically, the strength of evaluations and conclusions that are based on a profile of selected metabolite species, i.e., a panel of identified biomarkers, is improved compared to the isolated determination of the concentration of a single metabolite.

For immunological analysis, for example, the use of immunological reagents (e.g. antibodies), generally in conjunction with other chemical and/or immunological reagents, induces reactions or provides reaction products which then permit detection and measurement of the whole group, a subgroup or a subspecies of the metabolic product(s) of interest. Suitable immunological detection methods with high selectivity and high sensitivity (10-1000 pg, or 0.02-2 pmoles), e.g., Baldo, B. A., et al. 1991, A Specific, Sensitive and High-Capacity Immunoassay for PAF, Lipids 26(12): 1136-1139), that are capable of detecting 0.5-21 ng/ml of an analyte in a biofluid sample (Cooney, S. J., et al., Quantitation by Radioimmunoassay of PAF in Human Saliva), Lipids 26(12): 1140-1143).

In one embodiment of the invention, mass spectrometry is relied upon to detect metabolic products present in a given sample. In another embodiment of the invention, an ESI-MS detection device is relied upon to detect metabolic products present in a given sample. Such an ESI-MS may utilize a time-of-flight (TOF) mass spectrometry system. Quadrupole mass spectrometry, ion trap mass spectrometry, and Fourier transform ion cyclotron resonance (FTICR-MS) are likewise contemplated in additional embodiments of the invention.

In another embodiment of the invention, the detection device interfaces with a separation/preparation device or microfluidic device, which allows for quick assaying of many, if not all, of the metabolic products in a sample. A mass spectrometer may be utilized that will accept a continuous sample stream for analysis and provide high sensitivity throughout the detection process (e.g., an ESI-MS). In another embodiment of the invention, a mass spectrometer interfaces with one or more electrosprays, two or more electrosprays, three or more electrosprays or four or more electrosprays. Such electrosprays can originate from a single or multiple microfluidic devices.

In another embodiment of the invention, the detection system utilized allows for the capture and measurement of most or all of the metabolic products introduced into the detection device. In another embodiment of the invention, the detection system allows for the detection of change in a defined combination (“profile,” “panel,” “ensemble, or “composite”) of metabolic products.

Chemicals. Deuterium oxide (D₂O, 99.9% D) was purchased from Cambridge Isotope Laboratories, Inc. (Andover, Mass.). Trimethylsilylpropionic acid-d₄ sodium salt (TSP) was purchased from Sigma-Aldrich (analytical grade, St. Louis, Mo.).

Subject samples. Blood samples from PC patients (n=78) and healthy control subjects (n=48) were obtained from the Indiana University School of Medicine. The samples were obtained in two different batches within a span of one year, with the first batch consisting of 87 samples from 55 cancer patients and 32 controls and the second batch, 39 samples from 23 cancer patients and 16 controls. The controls in the first batch consisted of samples from 13 related subjects, and 19 unrelated subjects, and the controls in the second batch consisted of samples from 10 related subjects and 6 unrelated subjects; related subjects refer to familial genetically related volunteers (but not living in the same household as the PC patients), while the unrelated subjects refer to familial, non-genetically related volunteers. The mean age and range for cancer patients were 63 (48-86) years, while those for controls were 55 (39-86) years. Each blood sample was allowed to clot for 45 min and centrifuged at 1500 g for 10 min. The serum samples were separated, aliquoted into separate vials, frozen, and shipped over dry ice to Purdue University, where they were stored at −80° C. until analysis. Protocols approved by the Institutional Review Boards from both Indiana University School of Medicine and Purdue University were followed for collecting the blood samples; accordingly, the recruited subjects provided informed written consent.

¹H-NMR Spectroscopy All NMR experiments were carried out at 25° C. on a Bruker DRX 500 MHz spectrometer equipped with a cryogenic HCN triple resonance probe with triple-axis magnetic field gradients and operated using XWINNMR software version 3.5. The serum samples were thawed at room temperature and 570 μL, was transferred to 5 mm NMR tubes. A coaxial glass insert (OD 2 mm) containing 60 μL, of 0.012% TSP solution in D₂O was used as a chemical shift reference (δ=0.00 ppm) and field-frequency locking solvent. Two experiments were performed on each sample, one using the standard 1D NOESY (nuclear Overhauser effect spectroscopy) pulse sequence and the other using the CPMG (Carr-Purcell-Meiboom-Gill) pulse sequence. In both experiments, the water signal was suppressed by presaturation during the 3s recycle delay. Spectral widths, time domain data points, and the number of transients used were 6000 Hz, 32 K, and 32, respectively. An exponential weighting function corresponding to a line broadening of 0.3 Hz was applied to the free induction decay (FID), before Fourier transformation. Resulting spectra were phase and baseline corrected and subjected to further data and statistical analysis.

Statistical Analysis and Metabolite Identification. ¹H NMR spectra were aligned with reference to the alanine signal (1.46 ppm). After omitting the region between 4.00 to 6.00 ppm that contains the residual water and urea peaks, the other spectral regions between 0.50 to 9.00 ppm, were selected for data analysis. Each spectrum was normalized with reference to the total spectral sum excluding the lipid regions, and divided into variable spectral bins by manually selecting the regions with peaks and excluding those that had no peaks. Subsequently, fourteen regions were identified from the spectral bins corresponding to the metabolites alanine, asparagine, citric acid, creatinine, formate, glucose, glutamate, glutamine, histidine, isoleucine, lactate, phenylalanine, tyrosine, and valine. Identification of the peak regions for these metabolites was based on the literature data and the human metabolome database (HMBD). Wishart, D. S.; et al., HMDB: the human metabolome database. Nucleic Acids Res 2007, 35, D521-D526. See also Wishart, D. S.; et al., HMDB 3.0—The Human Metabolome Database in 2013, Nucleic Acids Research, 2013, Vol. 41, D801-D807, published online 17 Nov. 2012.

These selected metabolites were used for feature selection for developing classification model. Metabolites data for healthy related and healthy unrelated samples were combined to create one set of control samples, which were then used to compare with the data from cancer samples.

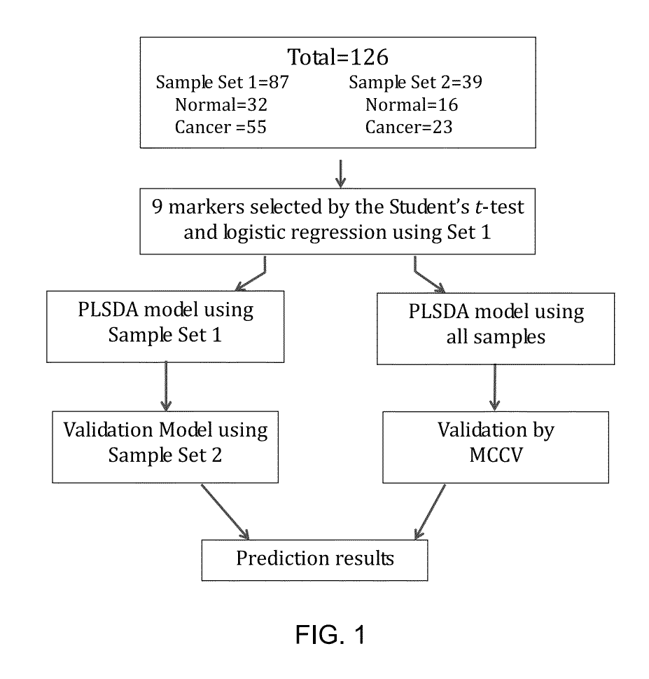

The general scheme used for statistical analysis is shown in FIG. 1. The first batch of 87 samples (training set) was used for metabolite selection and development of a statistical model, while a second independent batch of 39 samples (test set) was used for validation of the resulting model. L2-penalized logistic regression with a stepwise feature selection method was applied to the training set of samples. A binary variable identifying the PC patients and the controls was used as the response variable in the penalized logistic regression. L2 penalized logistic regression took into consideration the possible interaction among metabolites, and selected the metabolites that contributed to the classification. Nine metabolites, creatinine, glutamate, alanine, valine, histidine, lactate, glucose, glutamine and phenylalanine were thus selected by penalized logistic regression.

TABLE 1
¹H NMR Detected Metabolites That Contributed Significantly To
The Classification Of Pancreatic Cancer Patients And Healthy
Controls.

| | | | Fold |
|---|---|---|---|
| | Metabolite | p-value* | change** |
| 1 | Glutamate | 3.6 × 10⁻⁵ | 1.27 ± 0.06 |
| 2 | Formate | 0.002 | 1.37 ± 0.22 |
| 3 | Glucose | 0.020 | 1.11 ± 0.06 |
| 4 | Lactate | 0.005 | 1.15 ± 0.06 |
| 5 | Creatinine | 0.0002 | −1.23 ± 0.08 |
| 6 | Alanine | 0.0008 | −1.15 ± 0.05 |
| 7 | Glutamine | 0.015 | −1.13 ± 0.06 |
| 8 | Histidine | 0.003 | −1.16 ± 0.07 |
| 9 | Valine | 0.00005 | −1.18 ± 0.06 |

*Values determined from the Student's t-test.
**A negative value indicates a decrease in concentration.

The data were also analyzed using the Student's t-test to focus on metabolites that could contribute to the differentiation of PC patients from controls. Eight of the nine metabolites selected by penalized logistic regression had p<0.05; however, phenylalanine that was also selected had p=0.089. By contrast, formate, which was not selected by penalized logistic regression, had p=0.002. Based on the statistical significance, formate was included in modeling building in place of phenylalanine. Table 1, above, shows the list of metabolites along with their p-values and fold changes that were used to build a partial least squares discriminant analysis (PLS-DA) model. Ranges for the fold changes are based on an analysis of the standard errors of the mean values measured in the first batch of samples.

The NMR data corresponding to these 9 metabolites from the first batch of samples were imported to MATLAB (R2008a, Mathworks, Natick, Mass.) installed with the PLS TOOLBOX VERSION 4.0 (Eigenvector Research Inc., Wenatchee, Wash.). After log transformation and mean centering, a PLS-DA model was developed. Four latent variables (LV) were selected according to the root mean square error of cross validation (RMSECV)34-36 in leave-one-out cross validation. The PLS-DA model derived from the training set was then applied to the independent set of 39 samples collected in the second batch. The same procedure for peak integration was followed for the test set of samples before subjecting these samples to the PLS-DA model for validation. Predictive results for the validation set of samples in terms of sensitivity, specificity and area under the receiver operating characteristic (AUROC) curve were determined.

In order to further evaluate the robustness of the modeling, data from the two batches of samples were then combined. Monte Carlo Cross Validation (MCCV) was applied to the combined data to validate the accuracy of the PLS-DA model using the 9 metabolites. In every run, the combined data was divided into a training set of 87 samples and a validation set of 39 samples, i.e., the same size as the original batches of samples. The training and validation sets were randomly created for each of the three hundred iterations performed for MCCV. A PLS-DA model was constructed for each iteration using the training set with leave-one-out cross-validation, and the number of LVs was selected based on RMSECV as described above. The prediction results of the test set and the cross-validation prediction result of the training set were recorded for each iteration. To further assess model robustness, a second MCCV was conducted with same number (300) of iterations. Here, however, the class labels for the combined dataset were permuted for each iteration. Following the same MCCV process as before, a PLS-DA model was constructed on the training set and applied to the test set.

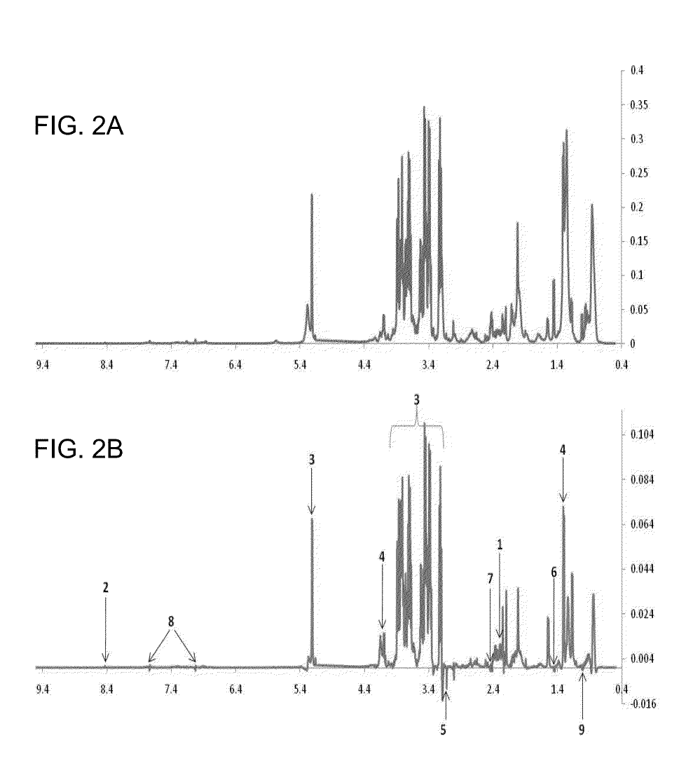

Referring now to FIG. 2, ¹H NMR spectra obtained using the NOESY pulse sequence were dominated by signals from macromolecules such as lipids and proteins. However, in the CPMG spectra, signals from macromolecules were effectively suppressed, which enabled clear visualization of the low molecular weight metabolites, and differences in metabolic features between PC and controls were clearly visible in the CPMG spectra (FIG. 2). FIG. 2A shows a ¹H NMR spectrum, obtained by averaging the spectra of pancreatic cancer samples in the training set. FIG. 2B shows a difference spectrum between the average spectrum of the pancreatic cancer and healthy controls from the training sample set. The numbered arrows indicate glutamate (1); formate (2), glucose (3), lactate (4), creatinine (5), alanine (6), glutamine (7), histidine (8), and valine (9). Accordingly, in this study, the metabolomics study of pancreatic cancer used NMR data obtained from the CPMG sequence.

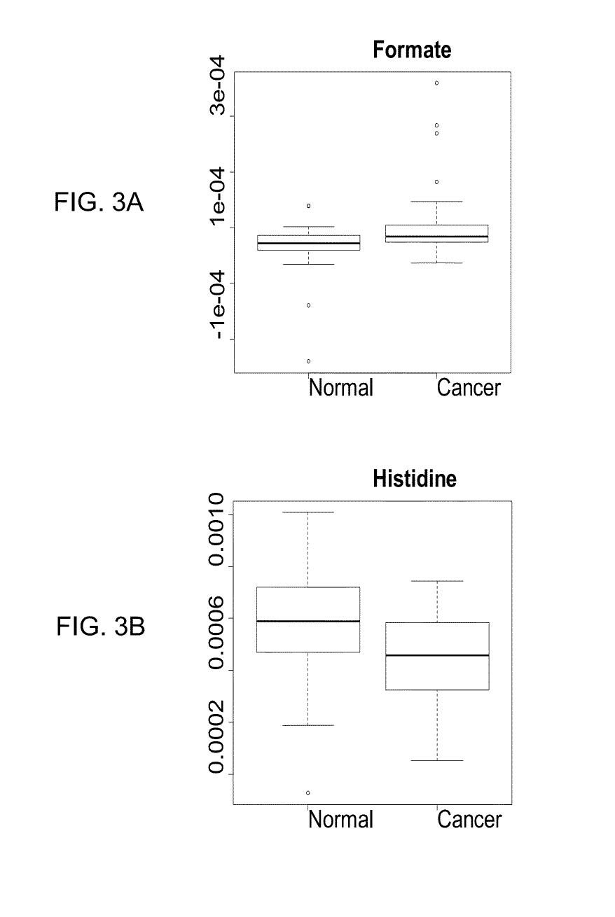

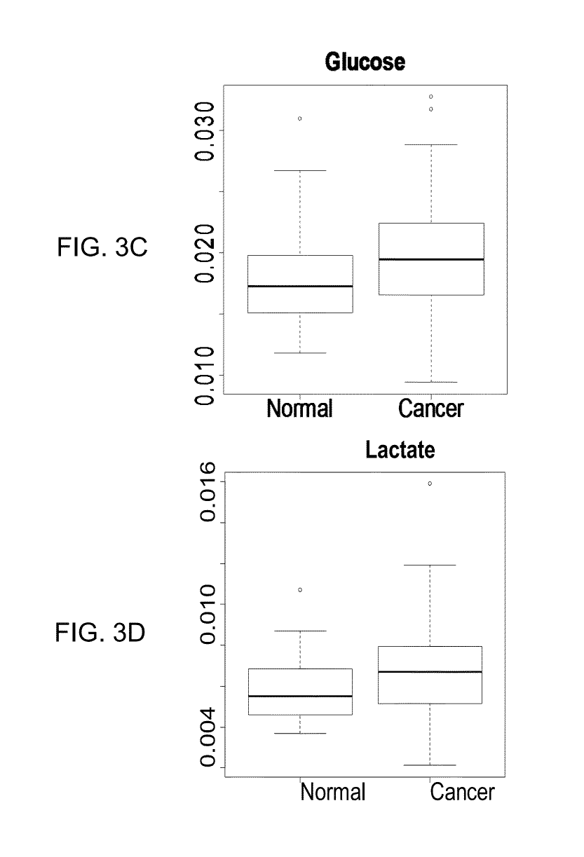

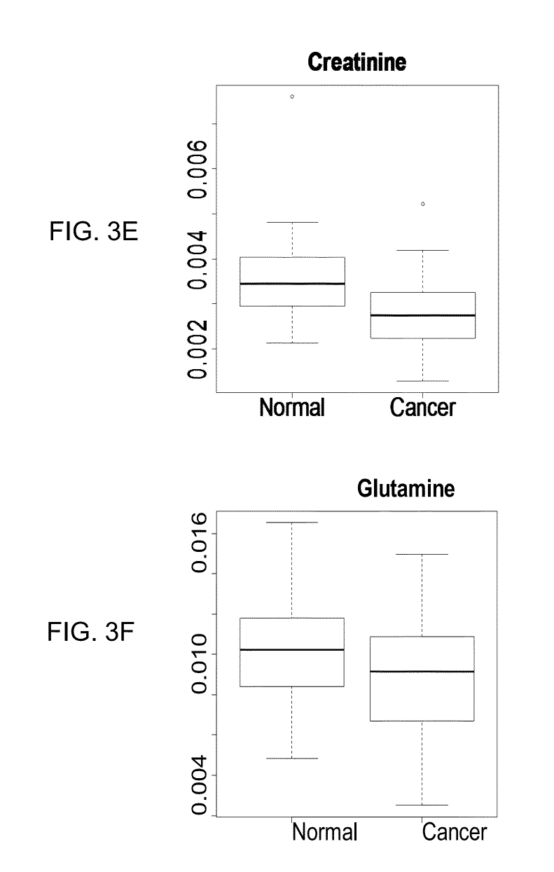

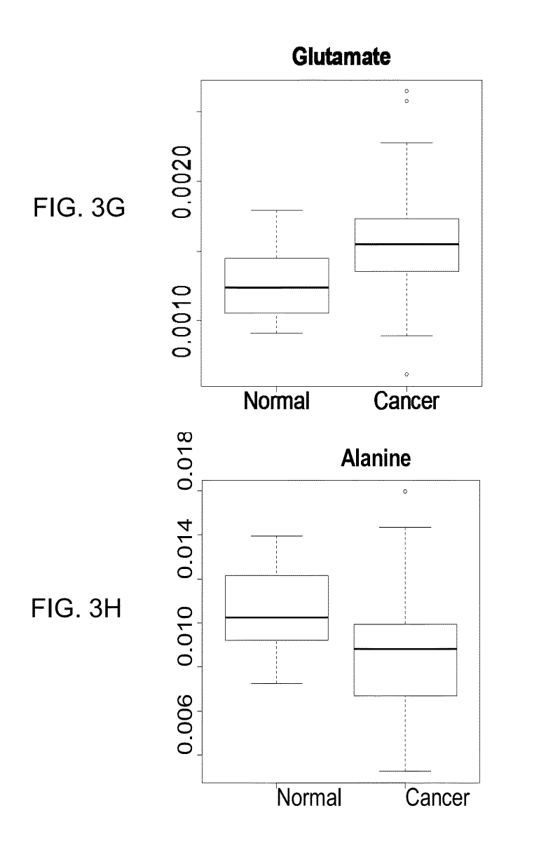

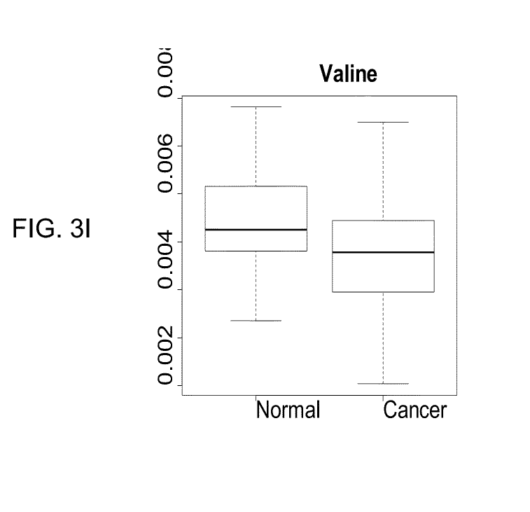

Biomarker selection and validation. The combination of univariate analysis (Student's t-test) and penalized logistic regression was used to select the metabolites of interest for classifying PC patients and controls. As a result of this analysis, nine highly ranked metabolites, which also showed significant difference between PC and controls, were selected for further analysis. FIG. 3A-FIG. 3I show the distribution of the relative concentrations of these metabolites in the PC patients and controls from the training set. All of these metabolites showed statistically significant changes in their levels, with p-values <0.05. FIG. 3A-FIG. 3I show box and whisker plots that show a comparison of concentration of selected metabolic biomarkers including formate, FIG. 3A, histidine, FIG. 3B, glucose, FIG. 3C, lactate, FIG. 3D, creatinine, FIG. 3E, glutamine, FIG. 3F, glutamate, FIG. 3G, alanine, FIG. 3H, and valine, FIG. 3I. Box-and-whisker plots showing the distribution of relative concentrations of the metabolites, are used for model building, in pancreatic cancer and normal subjects from the training set. The middle horizontal line in the box represents the median, the bottom and top boundaries represent the 25^{th} and 75^{th} percentiles, respectively. The lower and upper whiskers represent the 5^{th} and 95^{th} percentiles, respectively, and the open circles represent outliers.

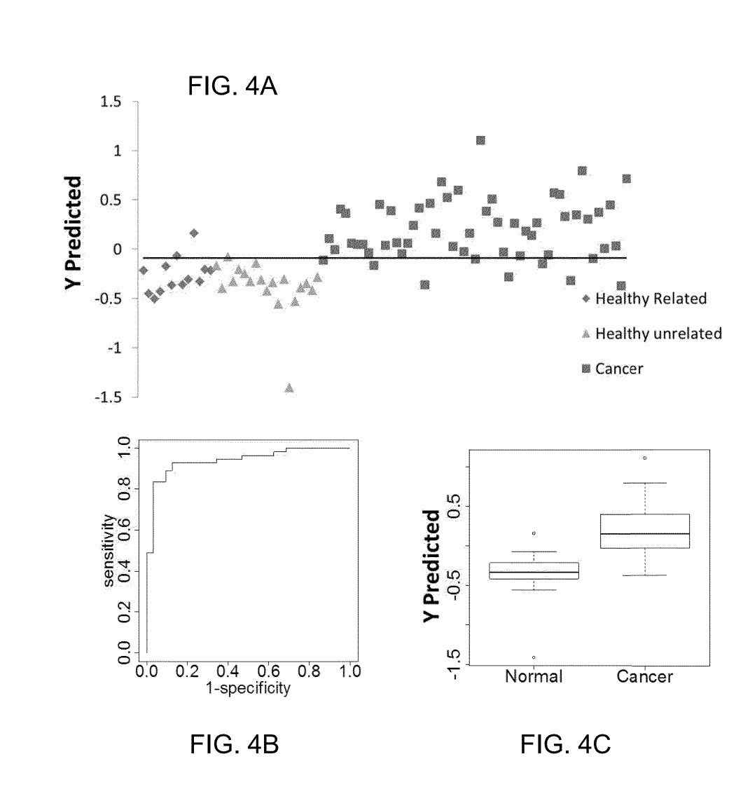

Five of these metabolites, alanine, glutamine, histidine, valine and creatinine were decreased in concentration in the cancer samples, while four metabolites, glutamate, glucose, formate and lactate, increased. Using these nine metabolites, the PLS-DA model was developed and validated following the steps shown in FIG. 1. The results of the PLS-DA model developed using the 87 training set of samples is shown in FIG. 4A-FIG. 4C. Distinctly separate clusters for PC and controls in the score plot (FIG. 4A). The model had an AUROC of 0.94 (FIG. 4B), with a sensitivity and specificity of 93% and 87%, respectively. The Y predicted scores for the model differed between PC and normal groups as shown in the Box-and-whisker plots (FIG. 4C).

Analysis of the PLS-DA scores for the two groups of healthy samples, familial genetically unrelated versus genetically related, showed that both sets of scores were very similar (e.g. mean values and standard deviation) with a p-value >0.4, indicating that there was no statistically significant difference in the metabolic profiles for the two types of control samples.

To establish the accuracy of the model for the detection of PC, the performance was then evaluated using an independent set of samples (23 PC; 16 controls). These samples had not been used for metabolite identification, feature selection, or development of the PLS-DA model.

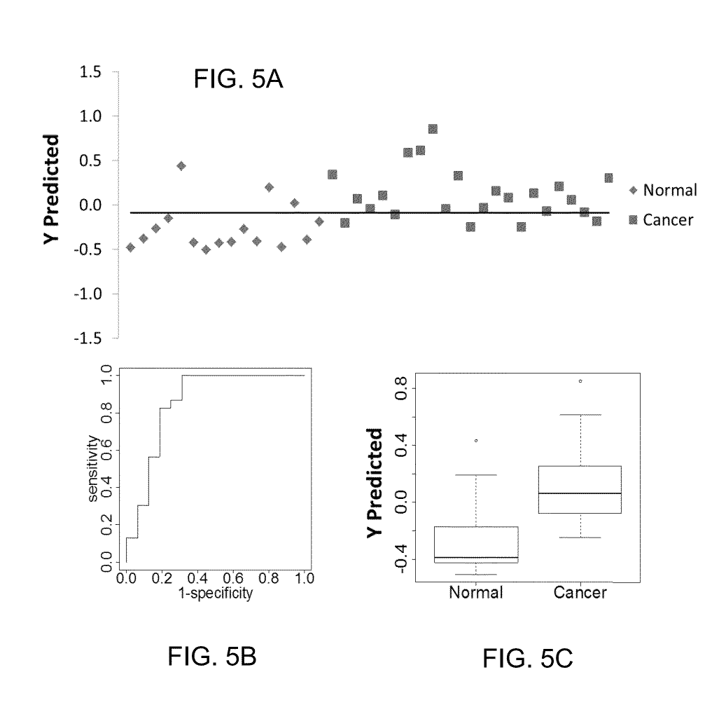

FIG. 5A-FIG. 5C show the performance of the PLS-DA model when applied to the test set of samples. Applying the PLS-DA model to this test set of samples resulted in an AUROC of 0.86 with a sensitivity and specificity of 87% and 75%, respectively. FIG. 5A-FIG. 5C and Table 2 show the MCCV results, which indicate the accuracy of the prediction model.

TABLE 2
Confusion Matrix Results For The PLS-DA Of 9 Biomarkers
Comparing Pancreatic Cancer Subjects (n = 78) and
Healthy Control Subjects (n = 48) using 300 MCCV
Iterations. The numbers in parentheses indicate the
results from class permutation analysis.

| | Total number of | Predicted class | |
|---|---|---|---|
| True class | samples | Normal | Cancer |
| Normal | 14400 (14400) | 12096 (5040) | 2304 (9360) |
| Cancer | 23400 (23400) | 5382 (9126) | 18018 (14274) |

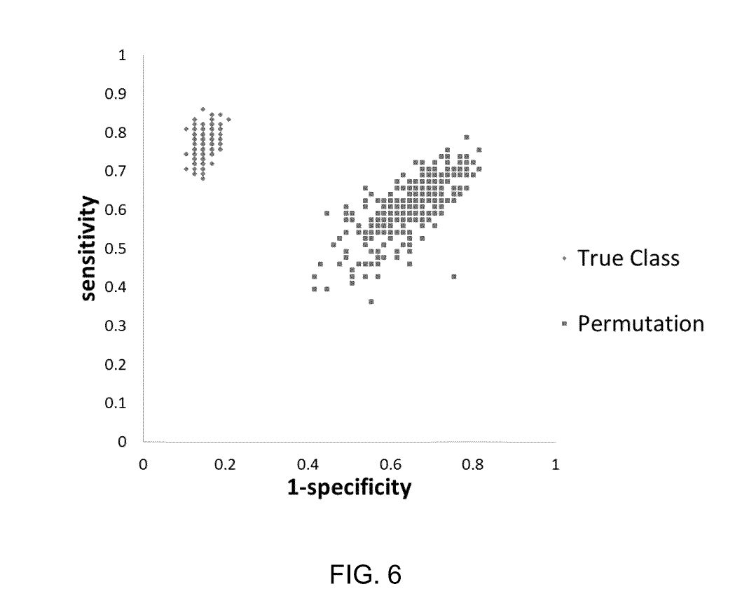

High sensitivity and specificity were displayed as seen from the ROC plot of the MCCV results, for nearly all 300 iterations (FIG. 6). The results for permutation cluster fall in the center of the space, indicating poor performance, as anticipated for a random assignment of class identity. The classification confusion matrix (Table 2) indicated a sensitivity of 77% and a specificity of 84% from the PLS-DA model in the first MCCV experiment, much better than a sensitivity of 61% and a specificity of 35% for the permutation iterations.

This study focused on the identification of metabolites associated with PC and the development of a metabolic profile for the classification of pancreatic cancer based on altered metabolite concentrations observed in serum. An analysis of serum metabolite signals derived from NMR measurements when combined with various univariate and multivariate statistical methods led to the identification of nine metabolite biomarker candidates that differentiated samples from pancreatic cancer patients from samples from healthy control subjects. The prediction model that was developed using these metabolites provided high classification accuracy in terms of both sensitivity and selectivity. Importantly, the model could be initially validated using an independent set of samples, and the classification accuracy was comparable to that obtained from the predication model.

With the aim of identifying biomarkers and validating the performance of the derived metabolites, we used two independent sets of serum samples from pancreatic cancer patients and healthy controls. The two sets of samples were obtained and the NMR experiments were performed during entirely different time periods. Major changes in metabolic profiles between PC and controls could be visualized through the altered mean concentrations of nine metabolites as indicated in Table 1.

When performing a method to detect the presence of pancreatic cancer in a subject, changes in concentration, compared to a comparable normal control subject or a predetermined value, of the nine identified metabolic biomarkers (or metabolite species) could be indicative of pancreatic cancer. For example, if a subject's biofluid biomarker concentrations shows a positive 1.21 to 1.33 fold increase of glutamate, or a positive 1.15 to 1.59 fold increase of formate, or a positive 1.05 to 1.17 fold increase of glucose, or a positive 1.09 to 1.21 fold increase of lactate, or a negative 1.15 to 1.31 fold decrease of creatinine, or a negative 1.10 to 1.20 fold decrease of alanine, or a negative 1.07 to 1.19 fold decrease of glutamine, or a negative 1.09 to 1.23 fold decrease of histidine, or negative 1.12 to 1.24 fold decrease of valine, or a combination thereof it may indicate a diagnosis of pancreatic cancer, see Table 3.

TABLE 3
Fold change ranges for various metabolites that could be indicative
of pancreatic cancer. Fold changes are concentrations' increase
or decrease compared to predetermined value.

| | Range of Concentration Fold Changes in |
|---|---|
| Metabolite | Metabolite species |
| Glutamate | 1.21 to 1.33 |
| Formate | 1.15 to 1.59 |
| Glucose | 1.05 to 1.17 |
| Lactate | 1.09 to 1.21 |
| Creatinine | −1.31 to −1.15 |
| Alanine | −1.20 to −1.10 |
| Glutamine | −1.19 to −1.07 |
| Histidine | −1.23 to −1.09 |
| Valine | −1.24 to −1.12 |

We first identified these metabolites as distinguishing markers of PC based on the combined regression and univariate analysis of the NMR data from the first, training set of samples. The PLS-DA based prediction model developed using these 9 metabolites was validated using the second, independent set of samples. The model consists of 10 coefficients (1 for each of the metabolites, plus a constant), which are determined from the training set. Each metabolite measurement is multiplied by its corresponding coefficient to generate a score for each sample. The score values for pancreatic cancer patients and healthy subjects are compared to determine the prediction accuracy of the model, as shown below in equation 1:

Score=β₀+β₁M₁+β₂M₂ . . . β₉M₉, (equation 1)

Where β is a coefficient determined by the PLS-DA modeling and M is a metabolite level or concentration.

It is clear from the internally validated model (FIG. 4) and its performance on the independent data set (FIG. 5) that the panel of metabolites markers is highly sensitive and promises a robust approach for distinguishing pancreatic cancer patients and healthy control subjects.

The same metabolites are identified using the advanced analytical techniques in healthy controls and virtually all types of diseases. Hence variation of an individual metabolite's level is of little value for classifying a specific disease, such as pancreatic cancer. In view of this, the statistical models developed using a group of highly ranked metabolites can provide applications for early stage diagnostic of disease.

Avoiding deleterious effects of metabolic contributions from confounding factors, unconnected with disease, is critical in the development of robust biomarkers. In this study, to minimize such effects arising from a major factor, diet, we used serum samples from overnight fasted patients as well as healthy controls; the suppression of the diet effect on the obtained results is reflected in the excellent prediction model and the close agreement of the independent, validation, data set with the model.

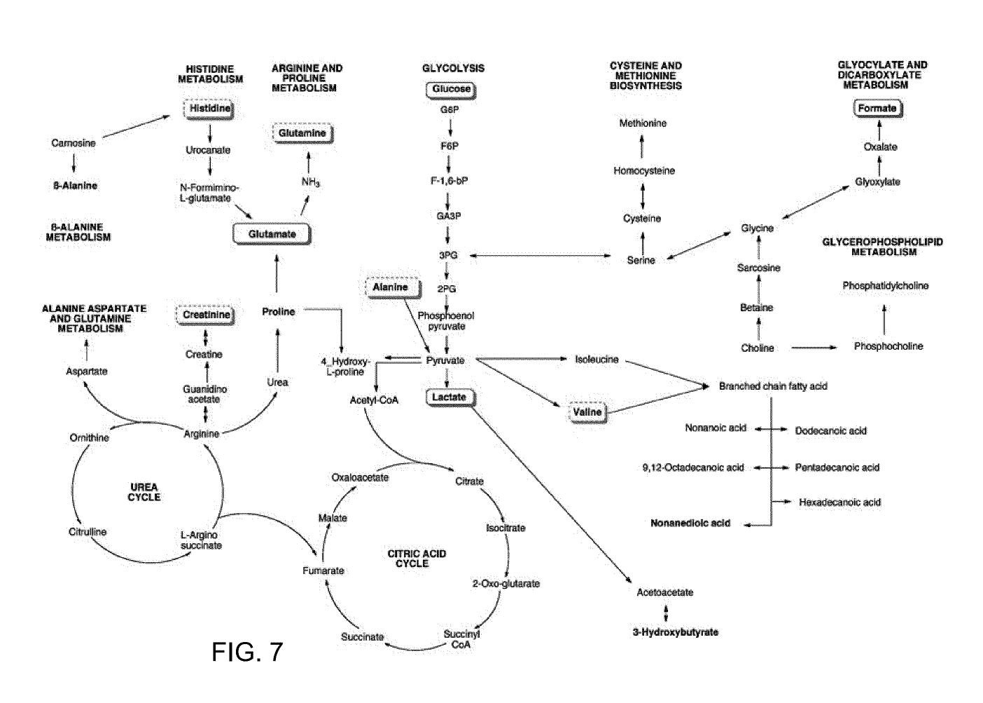

The metabolites identified in this study represent various biologically significant processes connected with pancreatic cancer development. FIG. 7, highlights the metabolic pathways associated with the metabolites that were altered in the pancreatic cancer samples compared to normal controls. Increased levels of glucose and lactate are consistent with increased glycolysis in malignancy; altered glycolysis is a common and long known phenomenon in growing cancer cells. Decreased levels of four amino acids, alanine, glutamine, histidine, and valine indicates increased demand for tumor growth and is consistent with numerous reports on cancer. In addition, we find increased levels for formate and glutamate, and decreased levels for creatinine in PC, which highlight the altered pathway associated with these metabolites.

What is claimed is:

1. A method comprising:
measuring the concentration of at least two metabolite species in a sample of a biofluid from a subject to be tested for pancreatic cancer, wherein the at least two metabolite species is a component of a panel of a plurality of metabolite species,
wherein a change in the concentration of the metabolite species is a characteristic that is associated with pancreatic cancer.

2. The method of claim 1 wherein the concentration of the metabolite species is normalized.

3. The method of claim 1, further comprising the step of:
comparing the measured concentration of the at least two metabolite species to a predetermined value calculated using a model based on concentrations of a plurality of the metabolite species that are components of the panel.

4. The method of claim 1, wherein the panel comprises two to nine metabolite species selected from the group consisting of alanine, creatinine, formate, glucose, glutamate, glutamine, histidine, lactate, and valine.

5. The method of claim 1 wherein the panel comprises metabolite species that have been identified by a plurality of methods selected from the group consisting of nuclear magnetic resonance (NMR) spectroscopy, gas chromatography-mass spectrometry (GC-MS), liquid chromatography-mass spectrometry (LC-MS), correlation spectroscopy (COSy), nuclear Overhauser effect spectroscopy (NOESY), rotating frame nuclear Overhauser effect spectroscopy (ROESY), LC-TOF-MS, LC-MS/MS, and capillary electrophoresis-mass spectrometry.

6. The method of claim 1 wherein the panel comprises metabolite species that have been identified by nuclear magnetic resonance (NMR) spectroscopy.

7. The method of claim 1 wherein the panel comprises metabolite species that have been identified by liquid chromatography-mass spectrometry (LC-MS).

8. The method of claim 1, wherein the biofluid is selected from the group consisting of blood, plasma, serum, sweat, saliva, sputum, and urine.

9. The method of claim 1, wherein the biofluid is serum.

10. A panel of metabolite species, the metabolite species selected from the group consisting of alanine, creatinine, formate, glucose, glutamate, glutamine, histidine, lactate, and valine.

11. The panel of claim 10, wherein the panel is provided in a diagnostic cassette.

12. The diagnostic cassette of claim 11, further comprising reagents for the detection of the metabolite species of the panel.

13. A kit for the analysis of a sample of a biofluid of a subject, comprising:
a. aliquots of standards of each compound of a panel of metabolite species;
b. an aliquot of an internal standard; and
c. an aliquot of a control biofluid.

14. The kit of claim 13, wherein the control biofluid is serum from a control source that is conspecific with the subject.

15. The kit of claim 13, wherein the panel consists of alanine, creatinine, formate, glucose, glutamate, glutamine, histidine, lactate, and valine.

16. The kit of claim 13, further comprising instructions for use.
